(19) United States
(12) Patent Application Publication
(10) Pub. No.: US 2007/0142892 A1
(43) Pub. Date: Jun. 21, 2007

(54) Self-expanding stent delivery system

(76) Inventors: Vipul Bhupendra Dave, Hillsborough, NJ (US); Diana Margarita Sanchez, Benardsville, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/304,256

(22) Filed: Dec. 15, 2005

(51) Int. Cl. A61F 2/06

(52) U.S. Cl. 623/1.11; 606/108

(57) ABSTRACT

Self-expanding stent delivery systems and methods having an introducer that receives a delivery catheter. The delivery catheter includes an outer body, an inner body and a stent loaded onto a stent bed within the inner body. The outer body receives the inner body with the stent loaded on the stent bed thereof. The outer body helps constrain the stent in its undeployed state in the stent bed until the stent is deployed by retraction of the outer body of the delivery catheter when the stent is identified as positioned across an intended treatment site. At least one anchoring mechanism provided on the inner body helps maintain the undeployed loaded stent appropriately in the stent bed during deployment. The at least one anchoring mechanism can include radiopaque material to increase fluoroscopic visualization of the stent during deployment, and the self-expanding stent can be a bio-absorbable material including drugs or other bio-active agents incorporated therein or provided thereon. The at least one anchoring mechanism can instead comprise a set of at least two bumpers between which the stent is loaded until deployed by retraction of the outer body of the delivery catheter when the stent has been appropriately positioned across an intended treatment site. After deployment of the stent at the intended treatment site, removal of the inner body and outer body of the delivery catheter and of the introducer occurs. Reliable and accurate emplacement of the stent across an intended treatment site is rendered more likely as a result.

### BACKGROUND OF THE INVENTION

1. Field of the Invention

The invention generally relates to a delivery system for emplacing a self-expanding stent within a vessel or other passageway of a patient.

2. Related Art

Stents for maintaining or restoring the patency of an anatomical passageway of a patient are commonly used to minimize the invasiveness otherwise associated with a surgical exposure of a treatment site. In the case of endovascular implantation of a stent into a blood vessel, percutaneous deployment is initiated by an incision into the vascular system of the patient, typically via the femoral or carotid artery. A tubular or sheath portion of an introducer is inserted through the incision and into the artery. A central lumen through the introducer provides a passageway through the patient's skin and artery wall into the interior of the artery. An outwardly tapered hub portion of the introducer remains outside the patient's body to prevent blood from leaking out of the artery along the outside of the sheath. A valve provided on the introducer is manipulated to block blood flow out of the artery through the introducer passageway. A distal end of a guide wire is passed through the introducer passageway and into the patient's vasculature. The guide wire is threaded through the vasculature until the inserted distal end of the guide wire extends just beyond the intended treatment site. The proximal end of the guide wire typically extends outside the introducer for manipulation by the medical practitioner.

Some self-expanding stents are known as “braided stents” having a plurality of rigid but flexible and elastic thread elements defining a radially expanding helix. Braided stents are typically held at a distal end of an outer catheter and pushed into position by an inner piston. Other types of self-expanding stents include alloys, such as Nitinol, having shape memory or superelastic characteristics. The shape memory characteristics allow deformation of the stent to facilitate insertion of the stent into the vessel, or other passageway, whereafter resumption of the original shape of the stent occurs when the stent is subjected to sufficient heat within the patient's body. The superelastic characteristics generally allow the metal to be deformed and restrained to facilitate insertion into the vessel, or other passageway, whereafter the restraint is then removed permitting the stent to return to its original undeformed shape.

One example of a self-expanding stent delivery system is U.S. Pat. No. 4,580,568 issued to Gianturco on Apr. 8, 1986. This reference discloses a delivery apparatus which uses a hollow sheath, like a catheter. The sheath is inserted into a patient's vessel and navigated therethrough so that its distal end is adjacent to the intended treatment site. The stent is then compressed to a smaller diameter and loaded into the sheath at the sheath's proximal end. A flat end pusher is inserted into the sheath and pushes the stent from the proximal end of the sheath to the distal end of the sheath. Once the stent is located at the distal end of the sheath adjacent to the intended treatment site, the sheath is pulled back while the pusher remains stationary, thereby exposing the stent and allowing the stent to expand within the vessel.

Delivering the stent the entire length of the catheter sheath can pose problems however, including damage to the vessel or the stent during deployment. Preloading the stent at the distal end of the catheter, as in U.S. Pat. No. 4,732,152 issued to Wallsten, et al. on Mar. 22, 1988, can pose other problems. Such problems include embedding of the stent in the interior surface of the distal end of the catheter or other conduit within the distal end of the catheter. Difficulty in sliding the catheter or other conduit over the preloaded stent can also occur during deployment even where actual embedding of the stent into the catheter or conduit does not occur.

Yet a further option for self-expanding stent delivery is set forth in U.S. Pat. No. 6,743,219 issued to Dwyer, et al. on Jun. 1, 2004, the contents of which are incorporated herein by reference, wherein an outer sheath receives an inner shaft and a stent. The inner shaft includes a flexible coiled portion to help the delivery device navigate tortuous vasculature. The inner shaft also includes a distal marker at the distal end of the inner stop, and a stop. A stent bed, on which the undeployed stent is positioned within the outer sheath, extends between the distal marker and the stop of the inner shaft. The delivery device positions the stent across an intended treatment site by aligning the distal marker and stop appropriately relative to the intended treatment site. Because the distal marker and the stop are provided with radiopaque materials, the alignment of the stent is readily monitored fluoroscopically. The stent maintains frictional contact with the interior surface of the outer sheath until the outer sheath is withdrawn to deploy the stent at the intended treatment site. The stop prevents the stent from sliding back, i.e, withdrawing, with the sheath and effectively “pushes” the stent out the distal end of the sheath as the sheath is withdrawn. In this manner, the stent is deployed as desired across an intended treatment site. While an effective alternative, the stent delivery system of Dwyer, et al., nevertheless still risks twisting or bunching of the stent during deployment or loading of the stent, that can hinder desirable emplacement of the stent across an intended treatment site.

In view of the above, a need exists for systems and methods that can more reliably and accurately emplace a self-expanding stent within a vessel or passageway of a patient.

### SUMMARY OF THE INVENTION

The various aspects of the systems and methods of the invention described herein provide a delivery system for reliably and accurately emplacing a self-expanding stent within a vessel or passageway of a patient.

In one embodiment, the delivery system comprises a delivery catheter working in complicity with a guide catheter or introducer in conventional manner. The delivery catheter further comprises an outer body, an inner body received within the outer body, and a self-expanding stent received on a stent bed along the inner body proximal to a distal end of the inner body so as to be between the inner body and the outer body in a loaded, undeployed state. The outer body thus acts as a sheath to protect and constrain the stent in its unexpanded state, while the inner body acts as a guide wire that assists in navigating the vasculature of a patient within which the stent is to be emplaced. At least one anchoring mechanism is provided on at least a proximal end of the stent bed. The at least one anchoring mechanism engages the loaded stent in its constrained state until deployment of the stent occurs and expansion of the stent results in the disengagement of the stent from the stent bed, the at least one anchoring mechanism and the inner body. The stent is a self-expanding stent comprised of a biostable polymer, bioabsorbable polymer or metal and can include drugs, bio-active agents and radiopaque markers. Radiopaque materials may be added to the anchoring mechanisms, and drugs or bio-active agents may be added to the stent and some, all or none of the anchoring mechanisms, as desired.

Alternatively, the at least one anchoring mechanism includes one anchoring mechanism provided at the proximal end of the stent bed and one anchoring mechanism provided at the distal end of the stent bed. In still other embodiments, the at least one anchoring mechanism includes anchoring mechanisms provided along the stent bed between the proximal end and the distal end of the stent bed. Of course, combinations of the above embodiments are also contemplated herein, as the artisan should readily appreciate. In any case, the at least one anchoring mechanism helps to maintain the stent in place between the inner body and the outer body during loading and deployment of the stent. In addition, the at least one anchoring mechanism provides support that helps minimize twisting or bunching of the stent during loading and deployment thereof. Radiopaque material can be added to the anchoring mechanisms in order to increase fluoroscopic visualization thereof. Accurate and reliable emplacement of the stent across an intended treatment site is thus enhanced. Drugs or other bio-active agents may be added to the stent and to some, all or none of the anchoring mechanisms, as desired.

In another embodiment, the at least one anchoring mechanism is a set of at least two bumpers located on the inner body, between which bumpers the stent is crimped when loaded onto the stent bed of the inner body prior to deployment. The bumpers preferably include radiopaque material (e.g., tungsten; tantalum; gold; barium sulfate; bismuth subcarbonate; iodine compounds; platinum; platinum/iridium or the like, and combinations thereof) so as to enhance visualization thereof during deployment of the stent. Drugs or other bio-active agents may be added to the stent and to some, all or none of the bumpers, as desired. As in the earlier described embodiment, after the delivery catheter is navigated through the vasculature and the stent is identified as positioned across the intended treatment site, the outer body is withdrawn. Withdrawal of the outer body permits the stent to disengage from the stent bed, the bumpers, and the inner body in general. Thereafter, the inner body is withdrawn and the stent is fully deployed as desired across the intended treatment site. The introducer/guide catheter is then withdrawn in conventional manner.

In yet another embodiment, the at least one anchoring mechanism is a set of at least two bumpers located on the inner body. One bumper is preferably located at the distal end of the stent bed and another bumper is located at the proximal end of the stent bed. Of course, other configurations are also contemplated herein including multiple bumpers along the stent bed with or without the bumpers at the proximal and distal ends of the stent bed, or bumpers in combination with anchoring mechanisms otherwise described herein, as the artisan will readily appreciate. The bumpers, or other anchoring mechanisms, preferably include radiopaque material to enhance visualization thereof during delivery of the stent. Drugs or other bio-active agents may be included in the stent and in some, all or none of the bumpers or other anchoring mechanisms, as desired.

In practice, the stent is loaded onto the stent bed of the inner body of the delivery catheter. The stent is oriented on the stent bed of the inner body so as to engage the at least one anchoring mechanism. The inner body with stent loaded thereon is then received within the outer body. The outer body thus protects and constrains the stent in its unexpanded state until deployment of the stent occurs by withdrawal of the outer body. Thereafter, the introducer/guide catheter and then the delivery catheter are introduced to the vasculature of a patient in conventional manner through an incision, for example, an incision in the femoral artery. The delivery catheter is then navigated through the vasculature of the patient to position the loaded stent across an intended treatment site. Flouroscopically visualizing the at least one anchoring mechanism helps identify when the loaded stent is located across the intended treatment site. Once the loaded stent is identified as positioned across the intended treatment site, then the outer body of the delivery catheter is withdrawn. Thereafter, the stent expands to disengage from the stent bed, the at least one anchoring mechanism and the inner body in general. The inner body is then withdrawn and the stent is fully deployed across the intended treatment site. The introducer/guide catheter is then withdrawn in conventional manner.

The above and other features of the invention, including various novel details of construction and combinations of parts, will now be more particularly described with reference to the accompanying drawings and claims. It will be understood that the various exemplary embodiments of the invention described herein are shown by way of illustration only and not as a limitation thereof. The principles and features of this invention may be employed in various alternative embodiments without departing from the scope of the invention.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates a generic schematic view of a self-expanding stent delivery system according to the invention.

FIG. 2 illustrates a cutaway view of the outer body, inner body and stent of the delivery catheter of FIG. 1.

FIG. 2A illustrates a stent bed having multiple anchoring mechanisms.

FIG. 2B illustrates a stent bed having a combination of anchoring mechanisms and bumpers.

FIG. 3 illustrates a self-expanding stent in expanded state in a vessel after withdrawal of the outer body of the delivery catheter according to the systems and methods of the invention

FIG. 4 illustrates an embodiment of a stent bed having a set of at least two bumpers according to the invention.

### DETAILED DESCRIPTION OF THE INVENTION

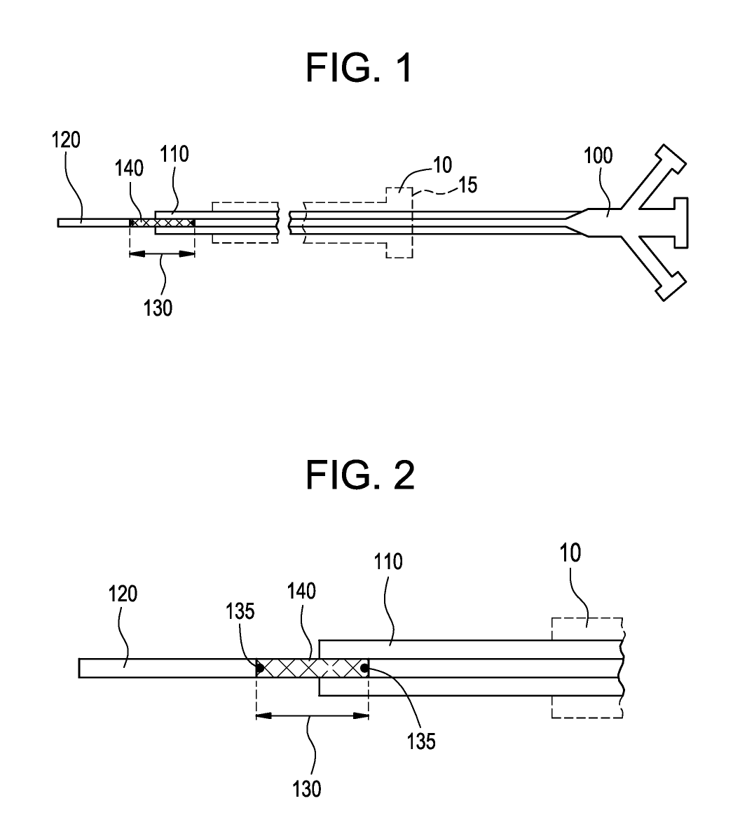

FIG. 1 illustrates a system for delivering a self-expanding stent to an intended treatment site in the vasculature of a patient, for example. The system generally comprises an introducer 10 (shown in dashed lines) and a delivery catheter 100. The delivery catheter 100 is insertable into the introducer 10 and secured by a valve 15, for example, in conventional manner, so as to restrict the delivery catheter 100 from undesirable movement when insertion and navigation of the delivery catheter 100 through the vasculature occurs. The valve 15 also minimizes, or ideally precludes, leakage of bodily fluids through the introducer where possible. The various components of the delivery system described herein are sized according to physiological and medical needs to accommodate a range of vessels or other anatomical passageways, as should be appreciated by the artisan.

Referring still to FIG. 1, the delivery catheter 100 further comprises an outer body 110, an inner body 120, and a stent bed 130, each having a respective proximal end and a distal end, wherein proximal is understood as closer to the operator and further from the patient and distal is understood as further from the operator and closer to the patient. The stent bed 130 is located proximal of the distal end of the inner body 120. In practice, a self-expanding stent 140 is loaded onto the inner body 120 of the delivery catheter 100 by positioning the stent 140 in the stent bed 130 of the inner body 120. The outer body 110 then receives the inner body 120 with the stent bed 130 loaded with the stent 140.

The interior diameter of the outer body 110 is thus dimensioned to accommodate the loaded inner body 120 and stent bed 130, wherein the artisan will appreciate that such dimensions are variable to accommodate various inner body, stent bed and stent configurations. The length of the outer body 110 is likewise dimensioned to accommodate the loaded inner body 120 and stent bed 130 so that the loaded stent 140 is fully received within the outer body. Of course, the artisan will appreciate that the dimensions of the outer body 110, as that of the inner body 120 and the stent bed 130 will vary according to the dimensions of the stent 140 that is to be loaded. The physiologic condition and site to be treated by the stent 140, and the judgment of the medical practitioner will contribute to determining appropriate dimensions of the various components comprising the various components of the systems and methods described herein. Because the outer body 110 receives the inner body 120 and stent bed 130 with a constrained self-expanding stent 140 loaded thereon, the outer body effectively acts as a sheath that helps maintain the stent 140 in its constrained state until deployment thereof occurs by eventual retraction of the outer body 110. The inner body 120 and the outer body 110 are generally comprised of known materials practiced in the art.

The stent can be comprised of bioabsorbable or biostable polymers with drugs or other bio-active agents and radiopaque markers incorporated therein. Drugs or other bio-active agents may be incorporated into or coated onto the stent in commonly used amounts or significantly greater amounts than in prior art stents. Likewise, radiopaque markers are provided in or on the stent. The combination of greater amounts of drugs or other agents for delivery from the device and the radiopaque markers improves the treatment of the targeted site, disease or condition and improves the visualization and placement of the device in the patient by the medical practitioner. The bioabsorbable polymeric materials that comprise the stent or other device according to the systems and methods of the invention are chosen based on several factors, including degradation time, retention of the mechanical properties of the stent or other device during the active drug delivery phase of the device, and the ability of the bioabsorbable materials to be processed into different structures and via different methods. Other factors, including cost and availability, may also be considered. Bioabsorbable polymeric materials that comprise the stent or other device according to the systems and methods of the invention may include shape memory polymers, polymer blends and/or composites that contribute to retaining the mechanical integrity of the device until drug delivery is completed.

Examples of bulk erosion polymers usable with the drug delivery devices according to the system and methods of the invention include poly (a-hydroxy esters) such as poly (lactic acid), poly (glycolic acid), poly (caprolactone), poly (p-dioxanone), poly (trimethylene carbonate), poly (oxaesters), poly (oxaamides), and their co-polymers and blends. Some commercially readily available bulk erosion polymers and their commonly associated medical applications include poly (dioxanone) [PDS suture], poly {glycolide) [Dexon suture], poly (lactide)-PLLA [bone repair], poly (lactide/glycolide) [Vicryl (10/90) and Panacryl (95/5) sutures], poly (glycolide/caprolactone (75/25) [Monocryl suture], and poly (glycolide/trimethylene carbonate) [Maxon suture].

Other bulk erosion polymers are also usable with the drug delivery devices according to the systems and methods of the invention such as tyrosine derived poly amino acid [examples: poly (DTH carbonates), poly (arylates), and poly (imino-carbonates)], phosphorous containing polymers [examples: poly (phosphoesters) and poly (phosphazenes)], poly (ethylene glycol) [PEG] based block co-polymers [PEG-PLA, PEG-poly (propylene glycol), PEG-poly (butylenes terphthalate)], poly (α-malic acid), poly (ester amide), and polyalkanoates [examples: poly (hydroxybutyrate (HB) and poly (hydroxyvalerate) (HV) co-polymers]. Other surface erosion polymers include poly (anhydrides) and poly (ortho esters).

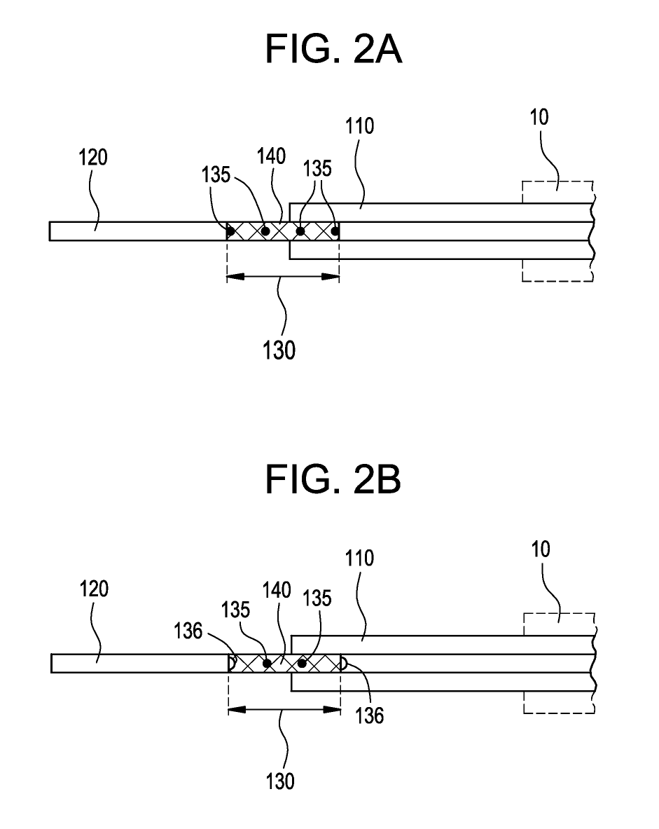

FIG. 2 illustrates in more detail, a cutaway view of the distal portion of the delivery catheter 100 of FIG. 1. As shown more clearly in FIG. 2, the stent bed 130 of the inner body 120 includes at least one anchoring mechanism 135. The at least one anchoring mechanism 135 is preferably located at the proximal end of the stent bed 130, when only one anchoring mechanism 135 is provided. When two or more anchoring mechanisms 135 are provided, at least one anchoring mechanism 135 is provided at the proximal end of the stent bed 130, and at least one other anchoring mechanism 135 is provided at the distal end of the stent bed 130. Alternatively, when two or more anchoring mechanisms 135 are provided, the anchoring mechanisms 135 may reside anywhere along the stent bed 130 between the proximal end and the distal end of the stent 140. In any case, the at least one anchoring mechanism 135 is engaged by the stent 140 when the stent 140 is loaded onto the stent bed 130 of the inner body 120. The at least one anchoring mechanism 135 thus helps to maintain the stent 140 in proper orientation and in its constrained state while deployment thereof occurs. The at least one anchoring mechanism 135 is comprised of polymeric or metallic materials and may include radiopaque material, such as tungsten; tantalum; gold; barium sulfate; bismuth subcarbonate; iodine compounds; platinum; platinum/iridium or the like and combinations thereof. Drugs or other bio-active agents may be added to the stent or to some, all or none of the anchoring mechanisms, as desired.

FIG. 2A illustrates a variation of the delivery system wherein the stent bed 130 includes multiple anchoring mechanisms 135 between the proximal end and the distal end of the stent bed 130. Although FIG. 2A shows four anchoring mechanisms 135, other amounts of anchoring mechanisms 135 are contemplated, including configurations wherein anchoring mechanisms 135 are provided at the proximal end and the distal end of the stent bed, as well as those wherein anchoring mechanisms 135 are not provided at the proximal end and distal end of the stent bed 130, or where an anchoring mechanism 135 is provided at the proximal end of the stent bed, and not at the distal end thereof, or vice versa.

FIG. 2B illustrates a variation of the delivery system wherein the stent bed 130 includes anchoring mechanisms 135 between the proximal end and the distal end of the stent bed 130. FIG. 2B further illustrates a bumper 136 at or immediately adjacent to the proximal end of the stent bed 130 and another bumper 136 at or immediately adjacent to the distal end of the stent bed 130. Such bumpers 136 are discussed in further detail below with respect to FIG. 4.

FIG. 4 illustrates another embodiment of the at least one anchoring mechanism, wherein like numerals are used. As shown in FIG. 4, the at least one anchoring mechanism 135 is a set of at least two bumpers 136. One bumper 136 is located at or immediately beyond the proximal end of the stent bed 130, and another bumper 136 is located at or immediately beyond the distal end of the stent bed 130. In another embodiment, at least one bumper 136 is located at or beyond the proximal end of the stent bed 130 and a plurality of additional bumpers 136 (not shown) are located along the stent bed 130 between the proximal and distal ends thereof. In this manner, the loaded stent 140 is oriented appropriately within the stent bed 130 in its constrained state while deployment thereof occurs.

Preferably, the at least one anchoring mechanism 135, is comprised of conventional polymeric or metallic material and may include radiopaque material, e.g., tungsten; tantalum; gold; barium sulfate; bismuth subcarbonate; iodine compounds; platinum; platinum/iridium or the like and combinations thereof. The radiopaque material enhances the fluoroscopic visualization of the location of the stent 140 positioned within the stent bed 130 of the delivery catheter 100 as navigation of a vessel occurs. Such visualization of the stent bed 130 via the radiopaque at least one anchoring mechanism 135 increases the accuracy and reliability of stent emplacement at an intended treatment site.

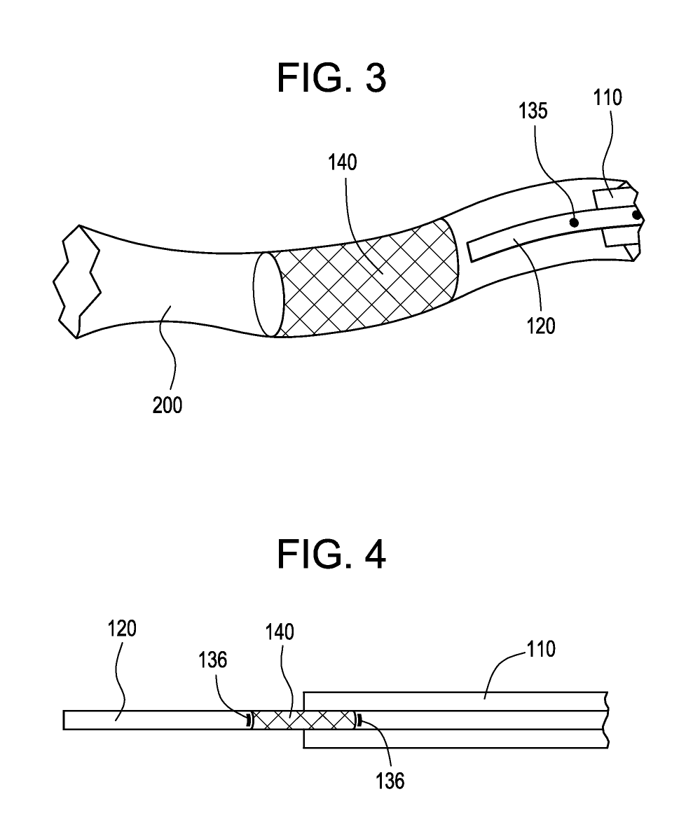

In practice, the stent 140 is loaded onto the stent bed 130 of the inner body 120 of the delivery catheter 100. The stent 140 is oriented on the stent bed 130 so as to engage the at least one anchoring mechanism 135. The inner body 120 with stent 140 loaded thereon in its constrained state is then received within the outer body 110. The outer body 110 thus protects and constrains the stent 140 in its constrained state. Thereafter, the introducer/guide catheter 10 and then the delivery catheter 100 with the loaded inner body 120 and stent bed 130 are introduced to the vasculature of a patient in conventional manner through an incision, for example, an incision in the femoral artery as the artisan should readily appreciate. The delivery catheter 100 is then navigated through the vasculature of the patient to position the loaded stent 140 across an intended treatment site. Flouroscopically visualizing the at least one anchoring mechanism 135 helps identify when the constrained stent 140 is located across the intended treatment site. Once the stent 140 is identified as positioned across the intended treatment site, then the outer body 110 of the delivery catheter 100 is withdrawn. Thereafter, the stent 140 expands to disengage from the stent bed 130, the at least one anchoring mechanism 135 and the inner body 120 in general. The inner body 120 is then withdrawn and the stent 140 is fully deployed across the intended treatment site. The introducer/guide catheter 10 is then withdrawn in conventional manner. FIG. 3 illustrates the emplacement of a stent 140 at an intended treatment site of a vessel 200 in this manner, whereby the outer body 110 has been retracted to enable the stent 140 to expand and disengage from the inner body 120. As should be appreciated from FIG. 3, once the stent 140 has expanded, the inner body 120 is readily retracted from the vessel 200. The introducer/guide catheter 10 and delivery catheter 100 are then otherwise withdrawn conventionally.

Where the at least one anchoring mechanism 135 is the set of bumpers 136, between which the stent 140 is loaded in its constrained state, then withdrawal of the outer body 110 permits the stent 140 to expand and disengage from between the bumpers 136 adjacent to the stent bed 130, and from the stent bed 130 and the inner body 120 in general. Thereafter, the inner body 120 is withdrawn and the stent 140 is fully deployed as desired across the intended treatment site. As before, the introducer/guide catheter 10 and delivery catheter 100 are then otherwise withdrawn in conventional manner.

The various exemplary embodiments of the invention as described hereinabove do not limit different embodiments of the systems and methods of the invention. The material described herein is not limited to the materials, designs or shapes referenced herein for illustrative purposes only, and may comprise various other materials, designs or shapes suitable for the systems and methods described herein, including metal, polymeric biostable or bioabsorbable self-expanding stents comprised of various materials, shapes and designs that may be crimped or otherwise retained by the various at least one anchoring mechanisms described herein, as should be appreciated by the artisan.

While there has been shown and described what is considered to be preferred embodiments of the invention, it will, of course, be understood that various modifications and changes in form or detail could readily be made without departing from the spirit or scope of the invention. It is therefore intended that the invention be not limited to the exact forms described and illustrated herein, but should be construed to cover all modifications that may fall within the scope of the appended claims.

What is claimed is:

1. A self-expanding stent delivery system comprising:
an introducer;
a delivery catheter insertable within the introducer, the delivery catheter further comprising:
an outer body having a proximal end and a distal end;
an inner body with at least one anchoring mechanism thereon, the inner body having a proximal end and a distal end an insertable within the outer body; and
a stent bed between the proximal end and the distal end of the inner body,
the stent bed having a proximal end and a distal end; and
at least one anchoring mechanism provided along the inner body and
engaging the self-expanding stent in a constrained state until deployment of the self-expanding stent occurs.

2. The self-expanding stent delivery system of claim 1, wherein the outer body comprises a sheath constraining the self-expanding stent until deployment thereof occurs.

3. The self-expanding stent delivery system of claim 1, wherein the at least one anchoring mechanism further comprises radiopaque material.

4. The self-expanding stent delivery system of claim 3, wherein the radiopaque material is comprised of at least one of tantalum, tungsten, gold, barium sulfate, bismuth subcarbonate, platinum, platinum/iridium, and iodine compounds.

5. The self-expanding stent delivery system of claim 3, wherein at least one of the self-expanding stent and the at least one anchoring mechanism further comprises a drug or bio-active agent.

6. The self-expanding stent delivery system of claim 3, wherein the at least one anchoring mechanism comprises an anchoring mechanism at a proximal end of the stent bed.

7. The self-expanding stent delivery system of claim 3, wherein the at least one anchoring mechanism comprises an anchoring mechanism at a proximal end and at a distal end of the stent bed.

8. The self-expanding stent delivery system of claim 7, wherein the at least one anchoring mechanism further comprises multiple anchoring mechanisms provided between the proximal end and the distal end of the stent bed.

9. The self-expanding stent delivery system of claim 8, wherein at least one of the anchoring mechanisms at the proximal end and the distal end of the stent bed is omitted.

10. The self-expanding stent delivery system of claim 3, wherein the at least one anchoring mechanism is a set of at least two bumpers, between which the stent is loaded until deployment thereof occurs.

11. The self-expanding stent delivery system of claim 10, wherein the set of at least two bumpers includes radiopaque material.

12. The self-expanding stent delivery system of claim 6, wherein the radiopaque material is comprised of at least one of tantalum, tungsten, gold, barium sulfate, bismuth subcarbonate, platinum, platinum/iridium, and iodine compounds.

13. The self-expanding stent delivery system of claim 3, wherein at least one of the self-expanding stent and the at least one anchoring mechanism further comprises a drug or bio-active agent.

14. The self-expanding stent delivery system of claim 10, wherein the set of at least two bumpers comprises at least one bumper at a proximal end of the stent bed and at least one bumper at a distal end of the stent bed.

15. The self-expanding stent delivery system of claim 10, wherein the at least two bumpers further comprise multiple bumpers between the proximal end and the distal end of the stent bed.

16. The self-expanding stent delivery system of claim 14, wherein at least one of the bumpers at the proximal end and the distal end of the stent bed is omitted.

17. The self-expanding stent delivery system of claim 8, wherein the at least one anchoring mechanism is a combination including at least one bumper and at least one non-bumper anchoring mechanism.

18. The self-expanding stent delivery system of claim 1, wherein the outer body further comprises an interior diameter accommodating receipt of the inner body and stent bed with the self-expanding stent loaded thereon.

19. The self-expanding stent delivery system of claim 1, wherein the self-expanding stent is comprised of polymeric materials.

20. The self-expanding stent delivery system of claim 19, wherein the self-expanding stent is further comprised of bioabsorbable materials.

21. The self-expanding stent delivery system of claim 20, wherein the self-expanding stent is further comprised of biostable polymers.

22. The self-expanding stent delivery system of claim 21, wherein the self-expanding stent is further comprised of metals.

23. The self-expanding stent delivery system of claim 22, wherein the self-expanding stent is further comprised of a combination of the polymers and the metals.

24. A method for delivering a self-expanding stent to an intended treatment site, the method comprising:
providing a self-expanding stent delivery system as in claim 1; and
loading the self-expanding stent onto the stent bed and engaging the at least one anchoring mechanism with the stent in its constrained state;
receiving the loaded stent and inner body into the outer body so as to maintain the stent in its constrained state by the outer body;
creating an incision into a vasculature of a patient;
inserting the introducer through into the patient through the vasculature of the patient through the incision;
inserting the delivery catheter with loaded self-expanding stent through the introducer and into the vasculature of the patient;
navigating the delivery catheter with loaded self-expanding stent through the vasculature of the patient to the intended treatment site;
withdrawing the outer body;
disengaging the stent from the inner body, the stent bed and the at least one anchoring mechanism by expansion of the stent; and
withdrawing the inner body, the stent bed and the at least one anchoring mechanism.

25. The method of claim 24, further comprising:
providing radiopaque material in the at least one anchoring mechanism; and
visualizing the location of the stent prior to withdrawal of the outer body by visualizing the at least one anchoring mechanism.

26. The method of claim 25, wherein the at least one anchoring mechanism is one anchoring mechanism at or adjacent a proximal end of the stent bed and one anchoring mechanism at or adjacent a distal end of the stent bed.

27. The method of claim 26, wherein the at least one anchoring mechanism is a set of at least two bumpers between which the stent is loaded.

28. The method of claim 27, wherein the set of at least two bumpers comprises at least one bumper at or adjacent a proximal end of the stent bed and at least one bumper at or adjacent a distal end of the stent bed.

29. A self-expanding stent delivery catheter comprising:
an outer body having a proximal end and a distal end;
an inner body having a proximal end and a distal end, the inner body being insertable within the outer body;
a stent bed between the proximal end and the distal end of the inner body,
the stent bed having a proximal end and a distal end and receiving the self-expanding stent therebetween the proximal end and the distal end of the stent bed; and
at least one anchoring mechanism along the inner body or stent bed, wherein
the at least one anchoring mechanism engages the self-expanding stent in a constrained state until deployment of the stent occurs.

30. The self-expanding stent delivery catheter of claim 29, wherein the outer body comprises a sheath constraining the self-expanding stent until deployment thereof occurs.

31. The self-expanding stent delivery catheter of claim 30, wherein the at least one anchoring mechanism further comprises radiopaque material.

32. The self-expanding stent delivery catheter of claim 31, wherein the radiopaque material is at least one of tantalum, tungstent, gold, barium sulfate, bismuth subcarbonate, platinum, platinum/iridium, and iodine compounds.

33. The self-expanding stent delivery catheter of claim 32, at least one of the self-expanding stent and the at least one anchoring mechanism further comprises a drug or bio-active agent.

34. The self-expanding stent delivery catheter of claim 33, wherein the at least one anchoring mechanism comprises an anchoring mechanism at a proximal end of the stent bed.

35. The self-expanding stent delivery catheter of claim 34, wherein the at least one anchoring mechanism comprises an anchoring mechanism at a proximal end and at a distal end of the stent bed.

36. The self-expanding stent delivery catheter of claim 34, further comprising multiple anchoring mechanisms between the proximal end and the distal end of the stent bed.

37. The self-expanding stent delivery catheter of claim 33, wherein the at least one anchoring mechanism is a set of at least two bumpers, between which the self-expanding stent is loaded until deployment thereof occurs.

38. The self-expanding stent delivery catheter of claim 37, wherein the set of at least two bumpers comprises at least one bumper at a proximal end of the stent bed and at least one bumper at a distal end of the stent bed.

39. The self-expanding stent delivery catheter of claim 38, further comprising multiple bumpers between the proximal end and the distal end of the stent bed.

40. The self-expanding stent delivery catheter of claim 39, wherein the at least one anchoring mechanism is a combination including at least one bumper and at least one non-bumper anchoring mechanism.

41. The self-expanding stent delivery catheter of claim 29, wherein the outer body further comprises an interior diameter accommodating receipt of the inner body and stent bed with the self-expanding stent loaded thereon.

42. The self-expanding stent delivery catheter of claim 29, wherein the self-expanding stent is comprised of a polymeric materials.

43. The self-expanding stent delivery catheter of claim 42, wherein the self-expanding stent is further comprised of bioabsorbable materials.

44. The self-expanding stent delivery catheter of claim 43, wherein the self-expanding stent is further comprised of biostable polymers.

45. The self-expanding stent delivery catheter of claim 44, wherein the self-expanding stent is further comprised of metals.

46. The self-expanding stent delivery catheter of claim 45, wherein the self-expanding stent is comprised of a combination of the polymers and the metals.
